(19) United States
(12) Patent Application Publication
(10) Pub. No.: US 2011/0054186 A1
(43) Pub. Date: Mar. 3, 2011

(54) PROCESS FOR EPROSARTAN INTERMEDIATE

(75) Inventors: Bandi Parthasaradhi Reddy, Hyderabad (IN); Kura Rathnakar Reddy, Hyderabad (IN); Rapolu Raji Reddy, Hyderabad (IN); Dasari Muraldihara Reddy, Hyderabad (IN); Medabalimi Peter Paul Raj, Hyderabad (IN)

(73) Assignee: HETERO RESEARCH FOUNDATION, Hyderabad, Andhrapradesh (IN)

(21) Appl. No.: 12/594,227

(22) PCT Filed: Mar. 31, 2008
(86) PCT No.: PCT/IN2008/000211

§ 371 Date: Oct. 1, 2009

(51) Int. Cl. C07D 409/06

(52) U.S. Cl. 548/315.1

(57) ABSTRACT

The present invention provides an improved process for preparation of (E)-α-[[2-butyl-1-[[4-(methoxycarbonyl)phenyl]methyl]-1H-imidazole-5-yl]methylene]-2-thiophene propanoic acid ethyl ester in high purity and in high yield. Thus, for example, 4-[[2-butyl-5-formyl-1H-imidazole-1-yl]methyl]benzoic acid methyl ester is reacted with ethyl 2-carboxy-3-(2-thienyl)propionate in the presence of piperidinium propionate in diisopropyl ether or a mixture of n-hexane and a solvent selected from toluene and cyclohexane, optionally purifying the crude compound to obtain (E)-α-[[2-butyl-1-[[4-(methoxy carbonyl)phenyl]methyl]-1H-imidazole-5-yl]methylene]-2-thiophene propanoic acid ethyl ester substantially free of 3-(2-thienyl)propanoic acid ethyl ester impurity.

### FIELD OF THE INVENTION

The present invention provides an improved process for preparation of eprosartan and its pharmaceutically acceptable acid addition salts thereof in high purity and in high yield.

### BACKGROUND OF THE INVENTION

U.S. Pat. No. 5,185,351 disclosed a variety of imidazolylalkenoic acid derivatives, processes for their preparation, pharmaceutical compositions in which they are present and use thereof. These compounds are angiotensin II receptor antagonists and are useful in regulating hypertension induced or exacerbated by angiotensin II, and in the treatment of congestive heart failure, renal failure, and glaucoma. Among them, eprosartan mesylate, chemically (αE)-α-[[2-n-Butyl-1[(4-carboxyphenyl)methyl]-1H-imidazol-5-yl]methylene-2-thiophenepropanoic acid monomethanesulfonate is a promising angiotensin II receptor antagonist useful in the treatment of hypertension, congestive heart failure and renal failure. Eprosartan is represented by the following structure:

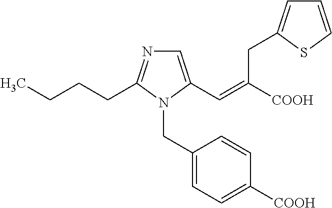

Processes for the preparations of eprosartan and related compounds were disclosed in U.S. Pat. No. 5,185,351, PCT publication No. 98/35963 A1 and European Patent No. 0973769 B1.

According to the U.S. Pat. No. 5,185,351 (herein after referred to as '351 patent), methyl 4-[[2-butyl-5-formyl-1H-imidazol-1-yl]methyl]benzoate is reacted with ethyl 2-carboxy-3-(2-thienyl)propionate, in the presence of a base, such as piperidine, in a suitable solvent, such as toluene, at a temperature of 80° C. to 110° C., preferably at 100° C., to give ethyl (αE)-α-[[2-n-Butyl-1-[[4-(methoxycarbonyl)phenyl]methyl]-1H-imidazol-5-yl]methylene-2-thiophene propionate, which is then hydrolyzed with a base such as sodium hydroxide to give eprosartan, which is further converted to eprosartan mesylate.

We have repeated the eprosartan synthetic procedure described in the '351 patent and found that relatively large amounts of impurities were obtained along with ethyl (αE)-α-[[2-n-Butyl-1-[[4-(methoxycarbonyl)phenyl]methyl]-1H-imidazol-5-yl]methylene-2-thiophene propionate when toluene is used as the solvent in the reaction between methyl 4-[[2-butyl-5-formyl-1H-imidazol-1-yl]methyl]benzoate and ethyl 2-carboxy-3-(2-thienyl)propionate in presence of piperidine at reflux temperature (100-120° C.), and hence the yield of the product is very poor (6-7%). If the above reaction is carried out in toluene without refluxing at 80-90° C. the reaction is not going forward.

In a specific run, we have found that ethyl (αE)-α-[[2-n-Butyl-1-[[4-(methoxycarbonyl)phenyl]methyl]-1H-imidazol-5-yl]methylene-2-thiophene propionate prepared by the above procedure, contained 65-70% of the decarboxylate impurity namely, ethyl 3-(2-thienyl)propionate, and 23-30% of some other impurities. It is observed that the decarboxylate impurity is further carried over to the next step, which is also converted to 3-(2-thienyl)propanoic acid during hydrolysis reaction with sodium hydroxide and found that it appeared as an impurity in eprosartan. The process described in the '351 patent also involves column chromatographic purifications.

Based on the aforementioned drawbacks, this process finds to be unsuitable for preparation of eprosartan at lab scale and commercial scale operations.

We have found that the formation of large amounts of the decarboxylate impurity in the above reaction is due to the degradation of 2-carboxy-3-(2-thienyl)propionate.

The '351 patent further described another process for preparation of eprosartan by using lithium derivatives such as n-butyl lithium. This process also suffers from drawbacks since it would be very difficult to handle lithium derivatives in large-scale scale operations, thereby making the process commercially not viable.

According to U.S. Pat. No. 6,172,237 B1, eprosartan is prepared by reacting 4-[[2-butyl-5-formyl-1H-imidazol-1-yl]methyl]benzoic acid or the bisulfite addition compound of 4-[[2-butyl-5-formyl-1H-imidazol-1-yl]methyl]benzoic acid with (2-thienylmethyl)propanedioic acid, mono-ethyl ester in a solvent (and/or solvent systems) selected from the group consisting of toluene, cyclohexane, cyclohexane:dichloroethane (12:5 or 1:1), cyclohexane:pyridine (12:5), and cyclohexane:ethyl acetate:pyridine (8:3:1) in the presence of piperidine as catalyst at reflux temperature at reduced pressure followed by hydrolysis of the intermediate ethyl ester (ethyl (αE)-α-[[2-n-butyl-1-[[4-(methoxycarbonyl)phenyl]methyl]-1H-imidazol-5-yl]methylene-2-thiophene propionate).

The yields of eprosartan obtained according to the processes described in the U.S. Pat. No. 6,172,237 B1 are very low, this is due to the yield loss resulted during the hydrolysis of methyl 4-[[2-butyl-5-formyl-1H-imidazol-1-yl]methyl]benzoate to obtain 4-[[2-butyl-5-formyl-1H-imidazol-1-yl]methyl]benzoic acid. Moreover, it is difficult to maintain the reaction at reflux under vacuum. The vacuum creates loss of solvent from reaction medium. So there is a need to add extra solvent to the reaction medium.

European Patent No. 0973769 provides processes for the preparation of eprosartan by using specific regioselective nitrogen-protecting reagents such as C₁₋₄-alkyl ester derivatives of acrylic acid.

The preparation of eprosartan as described in the European Patent No. 0973769 involves a lengthy process, the yields obtained in this process are very low and also the process is not satisfactory from purity point of view.

PCT patent application PCT/IN2006/000507 described a process for preparing eprosartan by reacting 4-[[2-butyl-5-formyl-1H-imidazole-1-yl]methyl]benzoic acid methyl ester with ethyl-2-carboxy-3-(2-thienyl)propionate in the presence of cyclohexane or n-hexane.

However, a need still remains for an improved process of preparing pure eprosartan solving the aforesaid problems associated with processes described in the prior art, which will be suitable for large-scale preparation, in terms of simplicity, chemical yield and purity of the product.

(E)-α-[[2-butyl-1-[[4-(methoxycarbonyl)phenyl]methyl]-1H-imidazole-5-yl]methylene]-2-thiophenepropanoic acid ethyl ester is the key intermediate in the preparation of eprosartan or pharmaceutically acceptable salt there of.

We have found that (E)-α-[[2-butyl-1-[[4-(methoxycarbonyl)phenyl]methyl]-1H-imidazole-5-yl]methylene]-2-thiophenepropanoic acid ethyl ester can be obtained in high yield with high purity when the reaction between 4-[[2-butyl-5-formyl-1H-imidazole-1-yl]methyl]benzoic acid methyl ester and ethyl-2-carboxy-3-(2-thienyl)propionate is carried out in a specific solvent system. The intermediate thus obtained can be used to prepare eprosartan and pharmaceutically acceptable salts thereof by known processes.

Thus, according to one object of the present invention there is provided a process for preparing (E)-α-[[2-butyl-1-[[4-(methoxy carbonyl)phenyl]methyl]-1H-imidazole-5-yl]methylene]-2-thiophenepropanoic acid ethyl ester a key intermediate in the preparation of eprosartan or a pharmaceutically acceptable salt in improved yields.

According to another object of the present invention, there is provided a novel purification method for (E)-α-[[2-butyl-1-[[4-(methoxycarbonyl)phenyl]methyl]-1H-imidazole-5-yl]methylene]-2-thiophenepropanoic acid ethyl ester, a key intermediate used for the manufacture of eprosartan or a pharmaceutically acceptable salt thereof.

### DETAILED DESCRIPTION OF THE INVENTION

According to one aspect of the invention there is provided an improved process for the preparation of (E)-α-[[2-butyl-1-[[4-(methoxy carbonyl)phenyl]methyl]-1H-imidazole-5-yl]methylene]-2-thiophenepropanoic acid ethyl ester of formula I:

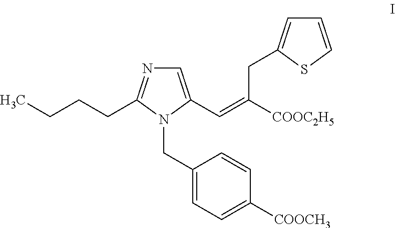

which comprises:
reacting 4-[[2-butyl-5-formyl-1H-imidazole-1-yl]methyl]benzoic acid methyl ester of compound of formula (II):

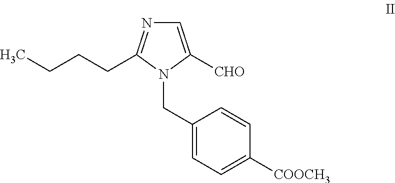

with ethyl-2-carboxy-3-(2-thienyl)propionate compound of formula (III):

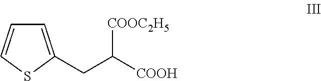

in diisopropyl ether, or a mixture of n-hexane and a solvent selected from toluene and cyclohexane.

The process may preferably be carried out in the presence of piperidine propionate. The process may also be carried out in the presence of piperidine.

If the mixture of solvents is used, the ratio of the quantity by volume of n-hexane to toluene or cyclohexane is preferably maintained between 1:1 to 7:1, more preferable ratio being 1.5:1 to 5:1.

The reaction is preferably carried out at 50-80° C. and more preferably at 60-75° C. under Dean-Stark apparatus to remove the water generated in the reaction.

According to another aspect of the present invention there is provided a process for purification of (E)-α-[[2-butyl-1-[[4-(methoxycarbonyl)phenyl]methyl]-1H-imidazole-5-yl]methylene]-2-thiophene propanoic acid ethyl ester containing 3-(2-thienyl)propanoic acid ethyl ester impurity in 10% or above to obtained (E)-α-[[2-butyl-1-[[4-(methoxy carbonyl)phenyl]methyl]-1H-imidazole-5-yl]methylene]-2-thiophene propanoic acid ethyl ester substantially free of 3-(2-thienyl)propanoic acid ethyl ester as an impurity, the said process comprises
i. Preparing a solution of (E)-α-[[2-butyl-1-[[4-(methoxy carbonyl)phenyl]methyl]-1H-imidazole-5-yl]methylene]-2-thiophene propanoic acid ethyl ester of formula (I) containing 3-(2-thienyl)propanoic acid ethyl ester impurity in a mixture of water and an alcohol at a p^{H} below 1.0
ii. Extracting the solution obtained in step (i) with diisopropyl ether to remove 3-(2-thienyl)propanoic acid ethyl ester impurity from the aqueous layer and
iii. Neutralising the aqueous layer with a base.

The solution in step (i) may be prepared by mixing (E)-α-[[2-butyl-1-[[4-(methoxycarbonyl)phenyl]methyl]-1H-imidazole-5-yl]methylene]-2-thiophene propanoic acid ethyl ester containing 3-(2-thienyl)propanoic acid ethyl ester impurity with water and an alcohol and then adding an acid such a HCl, H₂SO₄ till the p^{H} of the mass is decreased to below 1.0 preferably the p^{H} is decreased to 0.2 to 0.5. Preferably the acid used is sulphuric acid.

The neutralization in step (iii) is carried out by adding a base such as sodium hydroxide, sodium carbonate to p^{H} of 7 to 12. (E)-α-[[2-butyl-1-[[4-(methoxycarbonyl)phenyl]methyl]-1H-imidazole-5-yl]methylene]-2-thiophene propanoic acid ethyl ester substantially free of 3-(2-thienyl)propanoic acid ethyl ester impurity refers to (E)-α-[[2-butyl-1-[[4-(methoxy carbonyl)phenyl]methyl]-1H-imidazole-5-yl]methylene]-2-thiophenepropanoic acid ethyl ester containing less than 10% and preferably less than 5% of 3-(2-thienyl)propanoic acid ethyl ester of impurity.

(E)-α-[[2-butyl-1-[[4-(methoxycarbonyl)phenyl]methyl]-1H-imidazole-5-yl]methylene]-2-thiophenepropanoic acid ethyl ester obtained in substantially pure form may be further processed by conventional means and known procedures to obtain pure eprosartan or a pharmaceutical acceptable salt.

Thus for example the (E)-α-[[2-butyl-1-[[4-(methoxycarbonyl)phenyl]methyl]-1H-imidazole-5-yl]methylene]-2-thiophenepropanoic acid ethyl ester obtained according to the present invention may be isolated from the reaction mass and then hydrolyzed to obtain eprosartan; or (E)-α-[[2-butyl-1-[[4-(methoxy carbonyl)phenyl]methyl]-1H-imidazole-5-yl]methylene]-2-thiophenepropanoic acid ethyl ester may be hydrolyzed with a suitable base such as sodium hydroxide to obtain eprosartan. Eprosartan obtained is optionally converted into a pharmaceutically acceptable salt.

### EXAMPLES

### Example 1

Methyl 4-[[2-butyl-5-formyl-1H-imidazol-1-yl]methyl]benzoate (50 gm) and ethyl 2-carboxy-3-(2-thienyl)propionate (85 gm) are added to Diisopropyl ether (460 ml) under stirring at 25-30° C., the contents are heated to reflux for 1 hour under dean-stark to remove the traces of water. The reaction mass is cooled to 50° C.-60° C. and then a freshly prepared catalyst solution of propanoic acid (35.75 gm) and piperidine (14.5 gm) in diisopropyl ether (85 ml) is added drop wise. The resulting mass is heated to reflux at 65° C.-70° C. for 24 hours, cooled the reaction mass to 50-60° C. To the reaction mass a second lot of ethyl 2-carboxy-3-(2-thienyl)propionate (15 gm) is added, the contents are heated to reflux at 65°-70° C. for 48 hours and then distilled under vacuum. The resulting oily mass is stirred with toluene (166 ml) and water (83 ml), separated the layers and the organic layer is washed with 10% sodium chloride solution (80 ml) and then distilled under vacuum. The resulting oily mass is stirred with water (400 ml) and ethanol (300 ml), and then adjusted the pH of the mass to 0.4 with 33% H₂SO₄ solution (163 ml). The resulting aqueous layer extracted with Diisopropyl ether (2×200 ml), separated the layers and the aqueous layer is kept a side. Diisopropyl ether layer is subjected to distillation under vacuum to form oily residue, ethanol (130 ml) and water (170 ml) are added and the pH of the reaction mass is adjusted to 0.4 with 33% H₂SO₄ solution (110 ml). The resulting aqueous layer is extracted with diisopropyl ether (100 ml), The layers are separated and the organic layer is discarded. Both the aqueous layers are combined and the pH is adjusted to 11.5 with 50% aqueous NaOH (112 ml) at 25-35° C. The aqueous layer is extracted with toluene (2×300 ml), and the toluene is subjected to distillation under vacuum to obtain ethyl (αE)-α-[[2-n-butyl-1-[[4-(methoxycarbonyl)phenyl]methyl]-1H-imidazol-5-yl]methylene-2-thiophene propionate as oily residue. (80 gm., content of 3-(2-thienyl)propanoic acid ethyl ester impurity 4.5%).

### Example 2

Methyl 4-[[2-butyl-5-formyl-1H-imidazol-1-yl]methyl]benzoate (50 gm) and ethyl 2-carboxy-3-(2-thienyl)propionate (85 gm) are added to mixture of n-hexane (368 ml) and toluene (92 ml) under stirring at 25-30° C., the contents are heated to reflux for 1 hour under dean-stark to remove the traces of water. The reaction mass is cooled to 50° C.-60° C. and then a freshly prepared catalyst solution of propanoic acid (35.75 gm) and piperidine (14.5 gm) in a mixture of toluene (17 ml), n-hexane (68 ml) is added drop wise. The resulting mass is heated to reflux at 65° C.-70° C. for 24 hours, cooled the reaction mass to 50-60° C. To the reaction mass a second lot of ethyl 2-carboxy-3-(2-thienyl)propionate (15 gm) is added, the contents are heated to reflux at 65°-70° C. for 48 hours and then distilled under vacuum. The resulting oily mass is stirred with toluene (166 ml) and water (83 ml), separated the layers and the organic layer is washed with 10% sodium chloride solution (80 ml) and then distilled under vacuum. The resulting oily mass is stirred with water (400 ml) and ethanol (300 ml), and then adjusted the pH of the mass to 0.4 with 33% H₂SO₄ solution (163 ml). The resulting aqueous layer extracted with diisopropyl ether (2×200 ml), separated the layers and the aqueous layer is kept a side. Diisopropyl ether layer is subjected to distillation under vacuum to form oily residue, ethanol (130 ml) and water (170 ml) are added and the pH of the reaction mass is adjusted to 0.4 with 33% H₂SO₄ solution (110 ml). The resulting aqueous layer is extracted with diisopropyl ether (100 ml). The layers are separated and the organic layer is discarded. Both the aqueous layers are combined and the pH is adjusted to 11.5 with 50% aqueous NaOH (112 ml) at 25-35° C. The aqueous layer is extracted with toluene (2×300 ml), and the toluene is subjected to distillation under vacuum to obtain ethyl (αE)-α-[[2-n-butyl-1-[[4-(methoxycarbonyl)phenyl]methyl]-1H-imidazol-5-yl]methylene-2-thiophene propionate as oily residue. (90 gm, 3-(2-thienyl)propanoic acid ethyl ester impurity 2.5%).

### Example 3

Methyl 4-[[2-butyl-5-formyl-1H-imidazol-1-yl]methyl]benzoate (50 gm) and ethyl 2-carboxy-3-(2-thienyl)propionate (85 gm) are added to the mixture of n-hexane (368 ml) and toluene (92 ml) under stirring at 25-30° C., the contents are heated to reflux at 68-71° C. for 1 hour under dean-stark to remove the traces of water. The reaction mass is cooled to 50° C.-60° C. and then a freshly prepared catalyst solution of propanoic acid (35.75 gm) and piperidine (14.5 gm) in a mixer of toluene (17 ml), n-hexane (68 ml) is added dropwise. The resulting mass is heated to reflux at 68° C.-71° C. for 24 hours, cooled the reaction mass to 50-60° C. To the reaction mass ethyl 2-carboxy-3-(2-thienyl)propionate (15 gm) is added, the contents are heated to reflux at 68°-71° C. for 16 hours. The solvents are distilled off to obtain oily residue. Water (390 ml), 50% NaOH solution (100 gm) and ethanol (316 ml) are added, refluxed for 4 hours and cooled to 60-65° C. Washed reaction mixture with toluene (100 ml), separated the layers. Adjusted p^{H} of the aqueous layer to 5 with 6N HCl solution at 60° C. Cooled the reaction mass to 10-15° C., stir 1 hour and filtered to obtain (αE)-α-[[2-n-Butyl-1-[(4-carboxyphenyl)methyl]-1H-imidazol-5-yl]methylene-2-thiophenepropanoic acid as a solid (60 gm, 3-(2-thienyl)propanoic acid ethyl ester impurity; not detected, 3-(2-thienyl)propanoic acid impurity; not detected).

### Example 4

Methyl 4-[[2-butyl-5-formyl-1H-imidazol-1-yl]methyl]benzoate (50 gm) and ethyl 2-carboxy-3-(2-thienyl)propionate (85 gm) are added to mixture of n-hexane (306 ml) and cyclohexane (153 ml) under stirring at 25-30° C., the contents are heated to reflux for 1 hour under dean-stark to remove the traces of water. The reaction mass is cooled to 50° C.-60° C. and then a freshly prepared catalyst solution of propanoic acid (35.75 gm) and piperidine (14.5 gm) in a mixture of cyclohexane (28 ml), n-hexane (56 ml) is added drop wise. The resulting mass is heated to reflux at 65° C.-70° C. for 24 hours, cooled the reaction mass to 50-60° C. To the reaction mass a second lot of ethyl 2-carboxy-3-(2-thienyl)propionate (15 gm) is added, the contents are heated to reflux at 65° 70° C. for 48 hours and then distilled under vacuum. The resulting oily mass is stirred with toluene (166 ml) and water (83 ml), separated the layers and the organic layer is washed with 10% sodium chloride solution (80 ml) and then distilled under vacuum. The resulting oily mass is stirred with water (400 ml) and ethanol (300 ml), and then adjusted the p^{H} of the mass to 0.4 with 33% H₂SO₄ solution (163 ml). The resulting aqueous layer extracted with diisopropyl ether (2×200 ml), separated the layers and the aqueous layer is kept a side. Diisopropyl ether layer is subjected to distillation under vacuum to form oily residue, ethanol (130 ml) and water (170 ml) are added and the pH of the reaction mass is adjusted to 0.4 with 33% H₂SO₄ solution (110 ml). The resulting aqueous layer is extracted with diisopropyl ether (100 ml). The layers are separated and the organic layer is discarded. Both the aqueous layers are combined and the p^{H} is adjusted to 11.5 with 50% aqueous NaOH (112 ml) at 25-35° C. The aqueous layer is extracted with toluene (2×300 ml), and the toluene is subjected to distillation under vacuum to obtain ethyl (αE)-α-[[2-n-butyl-1-[[4-(methoxycarbonyl)phenyl]methyl]-1H-imidazol-5-yl]methylene-2-thiophene propionate as oily residue. (85 gm, 3-(2-thienyl)propanoic acid ethyl ester impurity 3.5%).

### Example 5

Methyl 4-[[2-butyl-5-formyl-1H-imidazol-1-yl]methyl]benzoate (50 gm) and n-hexane (368 ml) are added under stirring at 25-30° C., the contents are heated to reflux at 68-71° C. for 1 hour under dean-stark to remove the traces of water. The reaction mass is cooled to 50° C.-60° C. and then a freshly prepared catalyst solution of propanoic acid (35.75 gm) and piperidine (14.5 gm) in a mixer of toluene (17 ml), n-hexane (68 ml) is added dropwise. The resulting mass is heated to reflux at 68° C.-71° C., a solution of ethyl 2-carboxy-3-(2-thienyl)propionate (100 gm) in 92 ml toluene is added drop wise in 4 hours time and refluxed for 72 hours. The solvents are distilled off to obtain oily residue. Water (390 ml), 50% NaOH solution (100 gms) and ethanol (316 ml) are added, refluxed for 4 hours and cooled to 60-65° C. Washed reaction mixture with toluene (100 ml), separated the layers. Adjusted p^{H} of the aqueous layer to 5 with 6N HCl solution at 60° C. Cooled the reaction mass to 10-15° C., stirred for 1 hour and filtered to obtain (αE)-α-[[2-n-Butyl-1-[(4-carboxyphenyl)methyl]-1H-imidazol-5-yl]methylene-2-thiophenepropanoic acid as a solid (55 gm, 3-(2-thienyl)propanoic acid ethyl ester impurity; not detected, 3-(2-thienyl)propanoic acid impurity; not detected).

### Example 6

Methyl 4-[[2-butyl-5-formyl-1H-imidazol-1-yl]methyl]benzoate (50 gm) and ethyl 2-carboxy-3-(2-thienyl)propionate (85 gm) are added to the mixture of n-hexane (368 ml) and toluene (92 ml) under stirring at 25-30° C., the contents are heated to reflux for 1 hour under dean-stark to remove the traces of water. The reaction mass is cooled to 50° C.-60° C. and then a freshly prepared catalyst solution of propanoic acid (35.75 gm) and piperidine (14.5 gm) in a mixture of toluene (17 ml), n-hexane (68 ml) is added drop wise. The resulting mass is heated to reflux at 65° C.-70° C. for 24 hours, cooled the reaction mass to 50-60° C. To the reaction mass a second lot of ethyl 2-carboxy-3-(2-thienyl)propionate (15 gm) is added, the contents are heated to reflux at 65°-70° C. for 48 hours and then distilled under vacuum. The resulting oily mass is stirred with toluene (166 ml) and water (83 ml), separated the layers and the organic layer is washed with 10% sodium chloride solution (80 ml) and then distilled under vacuum. The resulting oily mass is stirred with water (400 ml) and ethanol (300 ml), and then adjusted the pH of the mass to 0.4 with concentrated HCl (100 ml). The resulting aqueous layer extracted with diisopropyl ether (2×200 ml), separated the layers and the aqueous layer is kept a side. Diisopropyl ether layer is subjected to distillation under vacuum to form oily residue, ethanol (130 ml) and water (170 ml) are added and the pH of the reaction mass is adjusted to 0.4 with concentrated HCl (28 ml). The resulting aqueous layer is extracted with diisopropyl ether (100 ml), the layers are separated and the organic layer is discarded. Both the aqueous layers are combined and the pH is adjusted to 11.5 with 50% aqueous NaOH (112 ml) at 25-35° C. The aqueous layer is extracted with toluene (2×300 ml), and the toluene is subjected to distillation under vacuum to obtain ethyl (αE)-α-[[2-n-butyl-1-[[4-(methoxycarbonyl)phenyl]methyl]-1H-imidazol-5-yl]methylene-2-thiophene propionate as oily residue. (72 gm, 3-(2-thienyl)propanoic acid ethyl ester impurity; 9%).

### Example 7

Methyl 4-[[2-butyl-5-formyl-1H-imidazol-1-yl]methyl]benzoate (50 gm) and ethyl 2-carboxy-3-(2-thienyl)propionate (85 gm) are added to mixture of n-hexane (368 ml) and toluene (92 ml) under stirring at 25-30° C., the contents are heated to reflux for 1 hour under dean-stark to remove the traces of water. The reaction mass is cooled to 50° C.-60° C. and then a freshly prepared catalyst solution of propanoic acid (35.75 gm) and piperidine (14.5 gm) in a mixture of toluene (17 ml), n-hexane (68 ml) is added drop wise. The resulting mass is heated to reflux at 65° C.-70° C. for 24 hours, cooled the reaction mass to 50-60° C. To the reaction mass a second lot of ethyl 2-carboxy-3-(2-thienyl)propionate (15 gm) is added, the contents are heated to reflux at 65°-70° C. for 48 hours and then distilled under vacuum. The resulting oily mass is stirred with toluene (166 ml) and water (83 ml), separated the layers and the organic layer is washed with 10% sodium chloride solution (80 ml) and then distilled under vacuum. The resulting oily mass is stirred with water (400 ml) and ethanol (300 ml), and then adjusted the pH of the mass to 0.4 concentrated HCl (100 ml). The resulting aqueous layer extracted with toluene (2×200 ml), separated the layers and the aqueous layer is kept a side. Toluene layer is subjected to distillation under vacuum to form oily residue, ethanol (130 ml) and water (170 ml) are added and the pH of the reaction mass is adjusted to 0.4 with concentrated HCl (28 ml). The resulting aqueous layer is extracted with toluene (100 ml), the layers are separated and the organic layer is discarded. Both the aqueous layers are combined and the pH is adjusted to 11.5 with 50% aqueous NaOH (112 ml) at 25-35° C. The aqueous layer is extracted with toluene (2×300 ml), and the toluene is subjected to distillation under vacuum to obtain ethyl (αE)-α-[[2-n-butyl-1-[[4-(methoxycarbonyl)phenyl]methyl]-1H-imidazol-5-yl]methylene-2-thiophene propionate as oily residue. (40 gm, 3-(2-thienyl)propanoic acid ethyl ester impurity; 10%).

### Example 8

Ethanol (150 ml) and water (200 ml) are added to ethyl (αE)-α-[[2-n-butyl-1-[[4-(methoxycarbonyl)phenyl]methyl]-1H-imidazol-5-yl]methylene-2-thiophene propionate (90 gm) (obtained in example 2 and 4) under stirring at 25-30° C., to the reaction mass added 50% NaOH solution (50 gm) and the contents are stirred at 55-60° C. for 4-5 hours, cooled to 25-30° C. The p^{H} of the reaction mass is adjusted to 5 with 6N HCl, then free flowing solid separates out. The reaction mass is cooled to 10-15° C. and stirred for 1 hour. Filtered the solid, washed with chilled water (110 ml) and then dried at 70-80° C. under vacuum to give 60 gm of eprosartan freebase (HPLC purity: 97%).

### Example 9

Acetic acid (240 ml) is added to eprosartan freebase crude (60 gm, purity 97%) under stirring at 25-30° C., the contents are heated to 80° C. until to form a clear solution and then stirred with activated carbon (6 gm) for 1 hour. Filtered the mass through hyflow bed, washed the bed with hot acetic acid (60 ml), the resulting filtrate is cooled to 25-30° C. and then methylene chloride (900 ml) is added drop wise to the mass at 25-30° C. and stirred for 24 hours at 25-30° C. The reaction mass is cooled to 0-5° C. and then stirred for 2 hours. Filtered the solid, washed with methylene chloride (52 ml) and then dried under vacuum to give 48 gm of pure eprosartan acetate (HPLC purity: 99.82%).

### Example 10

Eprosartan acetate (48 gm, obtained in example 9) is suspended in acetone (720 ml) at 25-30° C., the suspension is cooled to 0-5° C., methanesulfonic acid (33.92 gm) is added drop wise while maintaining the temperature at 0-5° C. The temperature of the mass is raised to 25-30° C. and stirred for 5 hours. Cooled the mass to 0-5° C., stirred for 1 hour, filtered the material and then suck dried. To the resulting wet cake added acetone (96 ml) and stirred for 30 minutes at 25-30° C. Filtered the material, washed with acetone (48 ml) and then dried to give 48 gm of pure eprosartan mesylate [HPLC Purity: 99.95%].

We claim:

1. A process for preparation of (E)-α-[[2-butyl-1-[[4-(methoxy carbonyl)phenyl]methyl]-1H-imidazole-5-yl]methylene]-2-thiophene propanoic acid ethyl ester of formula I: 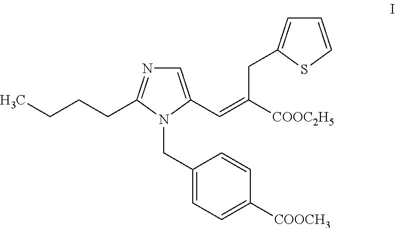
Which comprises
reacting 4-[[2-butyl-5-formyl-1H-imidazole-1-yl]methyl]benzoic acid methyl ester of compound of formula (II): 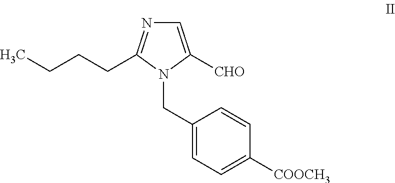
with ethyl-2-carboxy-3-(2-thienyl)propionate compound of formula (III): 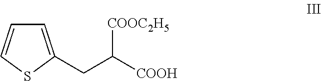
in diisopropyl ether or a mixture of n-hexane and a solvent selected from toluene and cyclohexane.

2. The process according to claim 1, the ratio of the quantity by volume of n-hexane to toluene or cyclohexane is maintained between 1:1 to 7:1.

3. The process according to claim 2, the ratio of the quantity by volume of n-hexane to toluene or cyclohexane is maintained between 1.5:1 to 5:1.

4. The process according to claim 1, the reaction is carried out in the presence of piperidine propionate.

5. The process according to claim 1, the reaction is carried out at 50 to 80° C.

6. The process for purification of (E)-α-[[2-butyl-1-[[4-(methoxy carbonyl)phenyl]methyl]-1H-imidazole-5-yl]methylene]-2-thiophene propanoic acid ethyl ester containing 3-(2-thienyl)propanoic acid ethyl ester impurity in 10% or above to obtain (E)-α-[[2-butyl-1-[[4-(methoxycarbonyl)phenyl]methyl]-1H-imidazole-5-yl]methylene]-2-thiophene propanoic acid ethyl ester substantially free of 3-(2-thienyl)propanoic acid ethyl ester as an impurity, the said process comprises
i. Preparing a solution of (E)-α-[[2-butyl-1-[[4-(methoxy carbonyl)phenyl]methyl]-1H-imidazole-5-yl]methylene]-2-thiophene Propanoic acid ethyl ester of formula (I) containing 3-(2-thienyl)propanoic acid ethyl ester impurity in a mixture of water and an alcohol at a p″ below 1.0
ii. Extracting the solution obtained in step (i) with diisopropyl ether to remove 3-(2-thienyl)propanoic acid ethyl ester impurity from the aqueous layer and
iii. Neutralising the aqueous layer with a base.

7. The process according to claim 6, the p″ of solution prepared in step (i) is 0.2 to 0.5.

8. The process according to claim 6, the base used in step (iii) is sodium hydroxide or sodium carbonate.
